(19) United States
(12) Patent Application Publication
(10) Pub. No.: US 2008/0008743 A1
(43) Pub. Date: Jan. 10, 2008

(54) Orally Dissolving Formulations of Memantine

(75) Inventors: Mahendra G. Dedhiya, Pomona, NY (US); Anil Chhettry, Holtsville, NY (US); Ranajoy Sarkar, Piscataway, NJ (US)

Correspondence Address: FOREST LABORATORIES, INC.;ATT: MICHAEL CIRAOLO, 48 MALL DRIVE, COMMACK, NY, 11725, US

(73) Assignee: Forest Laboratories Holdings Limited, Hamilton (BM)

(21) Appl. No.: 11/774,292

(22) Filed: Jul. 6, 2007

Related U.S. Application Data
(60) Provisional application No. 60/806,700, filed on Jul. 6, 2006.

(51) Int. Cl. A61K 9/70; A61K 31/13; A61P 25/00; A61P 43/00; A61K 9/22

(52) U.S. Cl. 424/443; 424/468; 514/662

(57) ABSTRACT

Orally dissolving formulations, e.g., tablets (ODTs) and films (ODFs) comprising memantine and methods of treating conditions, including childhood behavioral disorders and Alzheimer's disease, by administering orally dissolving formulations are provided. The orally dissolving formulations of the present invention may be used to treat various conditions, but is particularly suited to treat childhood behavioral disorders, such as autistic spectrum disorders or combined type Attention-Deficit/Hyperactivity Disorder (ADHD) and also to treat elderly patients suffering from Alzheimer's disease.

### CROSS-REFERENCE TO RELATED APPLICATIONS

This application claims priority under 35 U.S.C. §119, based on U.S. Provisional Application Ser. No. 60/806,700 filed Jul. 6, 2006, which is hereby incorporated by reference in its entirety.

### FIELD OF THE INVENTION

The present invention relates to orally dissolving formulations, e.g., tablets (ODTs) and films (ODFs) comprising memantine and methods of treating conditions, including childhood behavioral disorders and Alzheimer's disease, by administering orally dissolving formulations comprising memantine.

### BACKGROUND OF THE INVENTION

Various formulation techniques have been used to provide sustained and immediate release of pharmaceutically active agents. In many such formulations, a drug-containing or drug-bearing particle is coated by one or more release retardant layers or is dispersed within a continuous matrix such as a polymeric matrix. The coating layer or the matrix comprises a relatively insoluble material or materials, and the release of the drug is controlled by means of the resistance or permeability of the coating layer or matrix against the diffusion of the drug there through. The release of the drug from such formulations is driven by diffusion into the formulation, e.g., by the gradient of the drug concentration resulting from penetration of gastric fluid.

The use of orally dissolving formulations to administer pharmaceutical agents has also been disclosed. See, e.g., U.S. Pat. Nos. 3,784,390, 5,411,945, 5,980,882 and 6,001,392, the disclosures of which are hereby incorporated by reference in their entirety. Typically, the oral formulations contain a water-soluble polymer and other conventional excipients such as plasticizers and emulsifiers. However, the formulation composition will depend on the particular pharmaceutical agent and the desired formulation properties. For example, the formulation must be compatible with the pharmaceutical agent and also provide the necessary mechanical strength, taste-masking and dissolution properties.

Memantine (Namenda™) (1-amino-3,5-dimethyl adamantane), which is disclosed, e.g., in U.S. Pat. Nos. 4,122,193; 4,273,774; and 5,061,703, is a systemically-active uncompetitive NMDA receptor antagonist having low to moderate affinity for the receptor and strong voltage dependency and rapid blocking/unblocking kinetics. Memantine hydrochloride is currently available in the U.S. and in over 42 countries worldwide. It is approved for the treatment of moderate to severe Alzheimer's disease (AD) in the United States at a dose of up to 20 mg/day (5-10 mg BID). It has been hypothesized that memantine may not only be effective for the treatment of Alzheimer's disease (as well as Parkinson's and other neurological diseases), but may also be effective for the treatment of autism, Attention-Deficit/Hyperactivity Disorder (ADHD) and other autistic spectrum disorders.

Current dosing of memantine is twice a day using immediate release tablets. The tablet forms, however, are difficult to swallow and require the tablets to be coated to conceal its bitter taste. Moreover, the difficulties associated with tablets result in decreases patient compliance. Orally dissolving formulations of memantine are beneficial for many reasons. Their characteristic advantages such as administration without liquid, anywhere, anytime lead to their suitability in situations where patients have difficulty swallowing, such as children, the elderly and, particularly, those with neurological disorders.

There is an existing and continual need for release formulations containing memantine that provide reliable delivery and absorption of the active ingredient, while also providing a dosing regime that is straightforward and increases patient compliance.

### SUMMARY OF THE INVENTION

According to the present invention, it has now been found that memantine, and its salts, including the hydrochloride salt as well as other of its pharmaceutically acceptable salts can be formulated into orally dissolving formulations, e.g., tablets (ODTs) and films (ODFs). In addition, the present invention provides methods of treating conditions, including childhood behavioral disorders and Alzheimer's disease, by administering the orally dissolving formulations of the invention. The orally dissolving formulations of the present invention may be used to treat various conditions, but is particularly suited to treat childhood behavioral disorders, such as autistic spectrum disorders or combined type Attention-Deficit/Hyperactivity Disorder (ADHD) and also elderly patients suffering from Alzheimer's disease.

According to some embodiments, the present invention provides orally dissolving formulations that include at least one water soluble polymer and memantine.

According to other embodiments, the present invention provides methods for treating a patient in need thereof comprising administering to the patient an orally dissolving formulation comprising at least one water soluble polymer and memantine.

### BRIEF DESCRIPTION OF THE DRAWINGS

FIG. 1 shows the particle size distribution of uncoated and coated granules made with Pearlitol 160C.

### DETAILED DESCRIPTION OF THE INVENTION

The present invention relates to orally dissolving formulations, e.g., tablets (ODTs) and films (ODFs), comprising memantine and methods of treating conditions, including childhood behavioral disorders and Alzheimer's disease, by administering the orally dissolving formulations of the present invention.

According to some embodiments, the present invention provides orally dissolving formulations comprising at least one water soluble polymer and memantine or one of its pharmaceutically acceptable salts.

Memantine may preferably be used in the form of a pharmaceutically acceptable salt. Suitable salts of the compound include, but are not limited to, acid addition salts, such as those made with hydrochloric, methylsulfonic, hydrobromic, hydroiodic, perchloric, sulfuric, nitric, phosphoric, acetic, propionic, glycolic, lactic pyruvic, malonic, succinic, maleic, fumaric, maleic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. In a preferred embodiment, the salt is memantine hydrochloride (C₁₂H₂₁N.HCl, MW 215.77). The term “salts” can also include addition salts of free acids or free bases. All of these salts (or other similar salts) may be prepared by conventional means. All such salts are acceptable provided that they are non-toxic and do not substantially interfere with the desired pharmacological activity.

In addition, it is possible to use any salts and free base form of memantine including polymorphs, hydrates and solvates as well as amorphous forms of memantine. As used below in the present specification and claims “memantine” will be deemed to encompass both the free base and pharmaceutically acceptable salts thereof. In preferred embodiments of the invention, the active ingredient is memantine hydrochloride.

Memantine hydrochloride is a white, odorless substance that exists as needle-shaped crystals with a characteristic bitter taste. In some embodiments, the orally dissolving formulations, e.g., tablets (ODTs) and films (ODFs), may be formulated so that the taste of Memantine is masked. In further embodiments, the formulations should meet the FDA guidelines for disintegration (See e.g., Food and Drug Administration, Center for Drug Evaluation and Research, Guidance for Industry Orally Disintegrating Tablets April 2007) and provide a desired bioavailability. For example, the orally dissolving formulations of the present invention may disintegrate within 30 seconds and be bioequivalent to existing tablet and liquid formulations of memantine, e.g., immediate release formulations.

In some embodiments, the orally dissolving formulations of the present invention may include about 1% to about 50% (by weight) memantine. In preferred embodiments, the orally dissolving formulations of the present invention may include about 5% to about 30% (by weight) memantine.

In some embodiments, the orally dissolving formulations of the present invention may include a water-soluble polymer, a combination of two or more water-soluble polymers or a combination of a water-soluble polymer and a water-insoluble or-poorly-soluble polymer. Water soluble polymers that may be used in the orally dissolving formulations of the present invention include, but are not limited to, cellulose derivatives, synthetic polymers polyacrylates and natural gums. For example, the water soluble polymers used in the orally dissolving formulations of the present invention may include, but are not limited to, methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, agar, carrageenan, xanthan gum, tragacanth, guar gum, acacia gum, arabic gum, polyethylene glycol, polyethylene oxide, polyvinyl pyrrolidone, polyvinyl alcohol, cyclodextrin, carboxyvinyl polymers, sodium alginate, polyacrylic acid, methylmethacrylate or mixtures thereof. In exemplary embodiments, the concentration of the water-soluble polymer in the formulation may be about 20% to about 90% (by weight), preferably between about 40% to about 80% (by weight).

In some embodiments, the orally dissolving formulations of the present invention may comprise an excipient. Suitable excipients include, but are not limited to, microcrystalline cellulose, colloidal silicon dioxide, talc, starch, sorbitol, cyclodextrin or combinations thereof. In some embodiments, the excipient may include talc as anti-adhering agent.

In some embodiments, the orally dissolving formulations of the present invention may comprise a plasticizer. Suitable plasticizers include, but are not limited to, polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In exemplary embodiments, the concentration of the plasticizer in the formulation may be about 0 to about 30 wt %, preferably about 0 to about 10 wt % and more preferably about 0 to about 4 wt %.

In some embodiments, the orally dissolving formulations of the present invention may comprise an emulsifying agent. As used herein, emulsifying agents include both solubilizers and wetting agents. Suitable emulsifying agents include, but are not limited to, polyvinyl alcohol, sorbitan esters, cyclodextrins, benzyl benzoate, glyceryl monostearate, polyoxyethylene alkyl ethers, polyoxyethylene stearates, poloxamer, polyoxyethylene castor oil derivatives (Cremophor), hydrogenated vegetable oils, bile salts, polysorbates, ethanol or combinations thereof.

In other preferred embodiments, if present, the excipient is chosen to limit or avoid the formation of memantine adducts. As used herein, “adduct formation” refers to the formation of a compound with a particular formulation of a composition by a solid phase reaction. The general term “adduct” for a compound, also called an addition compound, results from the direct combination of two or more different compounds. For example, in the present invention, adduct formation (or other reducing sugars) may occur with formulations containing, for example, lactose (or other reducing sugars). Such adduct formation detracts from the efficacy of the product and increases the risks of other side effects.

In some embodiments, the orally dissolving formulations of the present invention may comprise a taste-masking agent. Generally, any natural or synthetic flavoring agent or sweetening agent known in the art may be used in the orally dissolving formulations of the present invention. For example, suitable taste-masking agents include, but are not limited to, essential oils, water soluble extracts, sugar, monosaccharides, oligosaccharides, aldose, ketose, dextrose, maltose, lactose, glucose, fructose, sucrose, mannitol xylitol, D-sorbitol, erythritol, pentitol, hexitol, malitol, acesulfame potassium, talin, glycyrrhizin, sucralose, aspartame, saccharin, sodium saccharin, sodium cyclamate, eugenyl formate aldehyde flavorings and combinations thereof.

Exemplary aldehyde flavorings that may be used include, but are not limited to acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, i.e., melonal (melon); 2-6-dimethyloctanal (green fruit); and 2-dodecenal (citrus, mandarin). In some embodiments, the taste-masking agents may include combination of acesulfame potassium and flavors. One skilled in the art with the benefit of the present disclosure will appreciate that other and further ingredients may be included in the orally dissolving formulations of the present invention. For example, a matrix-forming polymer permeation enhancer, substance for imparting mucoadhesive properties, or other auxiliary substances disclosed, for example, in U.S. Patent Publication No. 2005/0163830, the disclosure of which is hereby incorporated by reference in its entirety.

In some embodiments, the orally dissolving formulations of the present invention may comprise memantine that has been coated. The coating may be used to mask the taste of the memantine or change the dissolution profile of the active ingredient. Any coating suitable for use in pharmaceutical formulations may be used. See, e.g., R. C. Rowe in Materials used in Pharmaceutical Formulation, Blackwell Scientific Publications, Oxford, 1, 36 (1984), the disclosure of which is incorporated by reference herein in its entirety. Examples of suitable coating materials include polyethylene glycol, ethyl cellulose, methyl cellulose, hydroxypropyl methyl cellulose, acrylic resins, silicone elastomers, wax, fatty acids, polymethacrylate copolymers, Shellac, etc. In some embodiments, the coating may include between about 1% to about 75% of the formulation, preferably between about 10% to about 50% of the formulation.

In some embodiments, the orally dissolving formulations according to the present invention may include surfactants including, but not limited to, sodium docusate, polyoxyethylene ether, poloxamer, polysorbates (Tween), polyoxyethylene stearates, sodium lauryl sulfate, sorbitan esters and combinations thereof. If present, the surfactant may be included in the formulation from about 0.1% to about 10%, preferably between about 1% to about 5% (by weight). One skilled in the art, with the benefit of this disclosure, will understand that other components may be included to enhance one or more properties of the formulation. For example, the orally dissolving formulations according to the present invention may include disintegrating agents, antifoaming agents, antioxidants, buffering agents or coloring agents.

According to some embodiments, the present invention provides orally dissolving formulations for administration of memantine, or one of its pharmaceutically acceptable salts, to an individual in need thereof. For example, the orally dissolving formulations of the invention are suitable for the treatment of CNS disorders, including but not limited to the treatment of Alzheimer's disease, Parkinson's disease, AIDS dementia (U.S. Pat. Nos. 5,506,231, 5,061,703, and 5,614,560; see also Parsons et al., Neuropharmacology June 1999; 38(6):735-67), neuropathic pain (U.S. Pat. No. 5,334,618), cerebral ischemia (U.S. Pat. No. 5,061,703), epilepsy, glaucoma, hepatic encephalopathy, multiple sclerosis, stroke, depression (U.S. Pat. No. 6,479,553), tardive dyskinesia, malaria, Borna virus, Hepatitis C (U.S. Pat. Nos. 6,034,134 and 6,071,966). Additional pathologies for treatment of which memantine is suitable are disclosed in U.S. Pat. Nos. 5,614,560 and 6,444,702.

Memantine may not only be effective for the treatment of Alzheimer's disease (as well as Parkinson's and other neurological diseases), but may also be effective for the treatment of autism, ADHD and other autistic spectrum disorders. See U.S. application Ser. No. 11/234,764 (Published as US2006/0079582), the disclosure of which is hereby incorporated by reference in its entirety. The spectrum of childhood behavioral disorders include mental health problems such as anxiety disorders, Asperger's syndrome, ADHD, autistic spectrum disorders, autism, bipolar disorder, childhood disintegrative disorder, depression, disruptive behavior disorder, dyslexia, fragile X syndrome, learning disabilities, obsessive-compulsive disorder (OCD), oppositional defiant disorder, pervasive developmental disorder, reactive attachment disorder, Rett syndrome, separation anxiety disorder and Tourette's syndrome.

According to some embodiments, the present invention provides methods of administering memantine to a patient in need thereof comprising providing an orally dissolving formulation comprising at least one water soluble polymer and memantine. In some embodiments, the present invention provides methods for treating a disorder of the central nervous system, comprising administering to a patient in need thereof an orally dissolving formulation comprising an effective amount of memantine. In exemplary embodiments, the present invention provides methods of treating childhood behavioral disorders, such as autistic spectrum disorders or combined type Attention-Deficit/Hyperactivity Disorder (ADHD). In other exemplary embodiments, the present invention provides methods of treating Alzheimer's disease.

The present invention provides a formulation that when administered orally will dissolve to release (coated and/or uncoated) memantine. The formulation may release the memantine over a period of time that is determined by a number of different factors. These factors include the dimensions of the formulation, the concentration of the memantine, and how the memantine is dispersed throughout the formulation. For example, by varying the thickness and surface area of the formulations the rate of dissolution may be adjusted. A thick formulation will dissolve more slowly than an otherwise similar thin formulation and may be desirable to administer high dosages of memantine. In some embodiments, water soluble inert filler may be used in the formulation to increase the solubility of the memantine. One skilled in the art with the benefit of this disclosure will realize that the extent of memantine uptake can be controlled by the dissolution rate of the formulation. In addition, the memantine may be released from the formulation and swallowed so it is also taken up in the GI tract.

In exemplary embodiments, the orally dissolving formulations of the present invention may dissolve after less than about 30 seconds. In yet other exemplary embodiments, the orally dissolving formulations may dissolve after less than about 20 seconds.

In some embodiments, the memantine may be coated with a material to control the release of the memantine. Thus, the extent of memantine uptake can be controlled by the dissolution rate of the coated memantine. In other embodiments, the orally dissolving formulations of the present invention may include coated memantine or a mixture of coated and uncoated memantine. In exemplary embodiments, the coated memantine may be released from the formulation and swallowed so that uptake of the memantine occurs, partially or completely, in the GI tract.

### Definitions

The term “autism” refers to an individual demonstrating any one or all of the symptoms and characteristics associated with autism. Such individual may fit particular diagnostic criteria, such as Autistic Disorder, Asperger's Disorder, Atypical Autism or Pervasive Developmental Disorder, NOS (not otherwise specified), Rett's Disorder or Childhood Disintegrative Disorder, or the broader autism phenotype disorder or such individual may not fit a discrete diagnostic category at all. Due to the many presentations of the disease called autism, the present invention will use the term “autism” to refer to all of the above disorders.

As used herein, the terms “ODF,” “orally dissolving film,” and “orally disintegrating film” are used synonymously and mean that the film dissolves, melts, disintegrates, liquefies, etc. in the oral cavity such that substantially all of the memantine no longer remains in a formulation form.

The terms “ODT,” “orally dissolving tablet,” and “orally disintegrating tablet” are used synonymously and mean that the film dissolves, melts; disintegrates, liquefies, etc. in the oral cavity such that substantially all of the memantine no longer remains in a formulation form.

The “disintegration rate” is used herein to mean the amount of time that the film or tablet dissolves, melts, disintegrates, liquefies, etc. in the environment of an oral cavity such that substantially all of the memantine no longer remains in a formulation form, e.g., in saliva at pH greater than 5.

The “dissolution rate” is used herein to mean the amount of time that it takes for the memantine or pharmaceutically acceptable salt thereof to become bioavailable.

A “therapeutically effective amount” means the amount of a compound that, when administered to a mammal for treating a state, disorder or condition is sufficient to effect a treatment (as defined below). The “therapeutically effective amount” will vary depending on the compound, the disease and its severity and the age, weight, physical condition and responsiveness of the mammal to be treated. According to the instant invention, in one embodiment, a therapeutically effective amount of memantine is an amount effective to treat CNS disorders, including Alzheimer's disease or Parkinson's disease. In another embodiment, a therapeutically effective amount is an amount effective to treat neuropathic pain, or other painful conditions such as visceral hypersensitivity. Other uses include, but are not limited to, the treatment of dementia, depression, and neuropathic pain. The effective amount of the drug for pharmacological action, and therefore the capsule strength, depends on the disease itself, e.g., in Alzheimer's disease, the patient is initially given a 5 mg dose and the dosage is progressively increased to 10 mg twice a day. Additional doses evaluated in clinical trials include 40 mg/day. In the present invention, e.g., in Alzheimer's disease treatment the patient may be initially given 2.5 and increase to 80 mg.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

As used herein, the term “treat”, in all its verb forms, is used herein to mean to relieve or alleviate at least one symptom of a disorder in a subject, the disorder including for example, pain, Alzheimer's disease, vascular dementia, or Parkinson's disease. The term “treat” may mean to relieve or alleviate the intensity and/or duration of a manifestation of a disorder experienced by a subject in response to a given stimulus (e.g., pressure, tissue injury, cold temperature, etc.). For example, in relation to dementia, the term “treat” may mean to relieve or alleviate cognitive impairment (such as impairment of memory and/or orientation) or impairment of global functioning (activities of daily living, ADL) and/or slow down or reverse the progressive deterioration in ADL or cognition. Within the meaning of the present invention, the term “treat” also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the dementia is associated with a CNS disorder, including without limitation neurodegenerative diseases such as Alzheimer's disease (AD), Down's Syndrome and cerebrovascular dementia (VaD). The term “treatment” means the act of “treating” as defined above.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

To mask the bitter taste of memantine, particles of Memantine HCl were directly coated with methyl methacrylate-butyl methacrylate-dimethylaminoethyl methacrylate copolymer, Eudragit E (Degussa, Piscataway, N.J.) as the taste-masking polymer. Eudragit E is a cationic polymer and is soluble below a pH of 5 and swellable and permeable above pH of 5. Therefore, this polymer dissolves readily in stomach (pH 1-3), but resists dissolution in saliva pH greater than 5).

The drug particles (400 g) were loaded into the bowl of a Glatt Fluid Bed Coater (GPGC 3.1, Glatt Air Technique, Ramsey, N.J.). Eudragit dispersion was prepared according to manufacturer's instructions (Degussa, Piscataway, N.J.). Memantine drug substance was coated with the following conditions: Inlet Air Temperature 40 to 50° C.; Product Temperature 27 to 32° C.; Atomization pressure 1 to 2 Bars; Spray rate between 6-12 grams per minute; Target weight gain 5, 10, 15, 20, 25, 30, 35, 40, 45, 50% w/w. The resultingdrug product had up to a 50% weight gain of the taste-masking Eudragit polymer. The drug product composition is shown in Table 1.

TABLE 1
Composition of Direct Coated Polymer Dispersion

| Ingredient | Weight (g) |
|---|---|
| Methyl methacrylate-butyl methacrylate- | 125 |
| dimethylaminoethyl methacrylate copolymer | |
| (Eudragit E) | |
| Sodium Lauryl Sulfate | 12.5 |
| Stearic Acid | 18.75 |
| Mg Stearate | 43.75 |
| Water* | 1050.0 |
| Total | 1250 |

*Evaporates during the process

Under optical microscopic image analysis (Nikon Eclipse E600 Pol Polarizing Microscope equipped with a Nikon DXM1200F Digital Camera, Nikon, Melville, N.Y.) it was observed that even at 50% weight gain of the coating, large surface of the particles remain uncoated. The coated particles were tasted (n=2), without the individuals receiving a dose of the drug, to determine the effectiveness of taste masking. The bitter taste of the drug was readily perceived showing that the method did not mask the taste of memantine satisfactorily.

Although this method is widely used, it is not effective for direct coating of the unique needle-shaped particles of memantine because the end portions of the needles are especially difficult to coat and causes the drug to leach into the mouth. In addition, the coating process is difficult to control with needle-shaped crystals because they tend to fracture easily during processing, leading to creation of uncoated surfaces.

### Example 2

To overcome the difficulties encountered during direct coating of the Memantine HCl a process for coating the drug in a granular form was developed. Coating of granular drug particles, however, results in drug loading, i.e., the excipient to drug ratio is higher due to the use of additional excipients during granulation. Drug loading will affect the pharmokinetic parameters of a drug product, which may adversely affect the bioavailability of the final formulation. Consequently, granules with a particle size that is suitable for effective taste masking, while also providing a desired bioavailability must be identified.

Memantine, mannitol (Pearlitol 25C or 160C, Roquette America Inc., Keokuk, Iowa) and Povidone (Kollidon 90, BASF Corporation, Ledgewood, N.J.)) were dry mixed for 2 minutes in a Diosna High Shear Mixer/Granulator. Granulation was done by adding 300 g of water at an impeller speed of 300 rpm and chopper speed of 200 rpm followed by drying at 50° C. in an oven (Fisher Scientific). The dried granules were milled using a Fitz-Mill (The Fitzpatrick Company, Elmhurst, Ill.). The composition of the resulting Memantine granules is shown in Table 2.

TABLE 2
Composition of Memantine HCl Granules

| Ingredients | Function | Wt. in gms |
|---|---|---|
| Memantine HCl | Drug | 480 |
| Mannitol | Filler | 1440 |
| Polyvinyl | Binder | 80 |
| pyrrolidone | | |
| Water (evaporates | Solvent | 300 |
| during processing | | |
| Total | | 2000 |

The granules with a size of more than 150 microns, retained on #100 Sieve coated with a taste-masking polymer comprising methyl methacrylate-butyl methacrylate-dimethylaminoethyl methacrylate copolymer (such as Eudragit E PO). See Table 3.

TABLE 3
Composition of Taste-Masking Coating Dispersion

| Ingredient | Weight(g) |
|---|---|
| Methyl methacrylate-butyl methacrylate- | 400.0 |
| dimethylaminoethyl methacrylate copolymer (Eudragit | |
| E PO) | |
| Sodium Lauryl Sulfate | 40.0 |
| Stearic Acid | 60.0 |
| Mg Stearate | 140 |
| Water | 3360 |
| Total | 4000 |

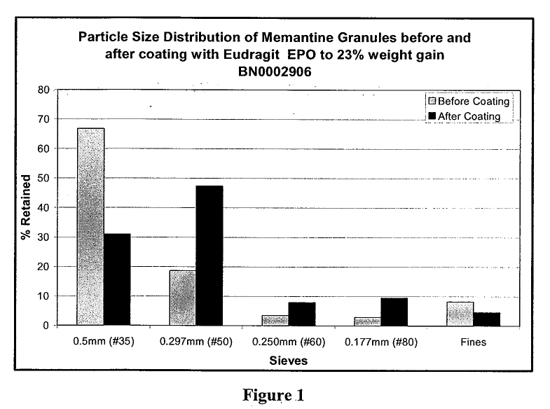

A Eudragit dispersion was prepared according to manufacturer's instructions (Degussa, Piscataway, N.J.). The particles were then coated to taste mask the particles using a Glatt Fluid Bed Coater (GPGC 3.1, Glatt Air Technique, Ramsey, N.J.) with the following conditions: Inlet Air Temperature 40 to 50° C.; Product Temperature 27 to 32° C.; Atomization pressure 1 to 2 Bars; Spray rate between 6-12 grams per minute; and Target weight gain 16, 32 and 36%. The resulting composition of the taste masked drug product is shown in Table 4. The particle size distribution of uncoated and coated granules made with Pearlitol 160C are shown in FIG. 1.

TABLE 4
Composition of Coated Taste-Masked Granules

| Ingredients | % w/w | Ranges % w/w |
|---|---|---|
| Memantine HCl | 17.6 | 5-75 |
| Mannitol | 52.7 | 15-75 |
| Polyvinyl pyrrolidone | 2.9 | 2-10 |
| methyl methacrylate-butyl | 16.7 | 5-30 |
| methacrylate- | | |
| dimethylaminoethyl | | |
| methacrylate copolymer | | |
| (Eudragit E PO) | | |
| Sodium Lauryl Sulfate | 1.7 | 0.5-3 |
| Stearic Acid | 2.5 | 0.5-5 |
| Mg Stearate | 5.9 | 1-7 |
| Total | 100 | 100 |

The coated particles were tasted (n=2), without the individuals receiving a dose of the drug, to determine the effectiveness of taste masking. The bitterness of the drug was not noticeable upon tasting showing that the coated granules were effectively taste-masked with Eudragit E dispersion. Thus, the characteristic bitter taste of memantine was effectively taste-masked using the described granulation approach.

### Example 3

Complexation with agents such as cyclodextrins may be used, alone or in combination with the granulation method described in Example 2, to reduce the bitter taste of orally dissolving formulations of memantine, e.g., tablets (ODTs) and films (ODFs). Memantine HCl was complexed with hydroxypropyl β-cyclodextrin (HPBCD) in 1:2 molar ratio, e.g., 10 mg of memantine was complexed with 130 mg of HPBCD and then compressed into tablets of suitable size or incorporated into films. For example, films were prepared by dissolving polyvinyl pyrrolidone in ethanol followed by the addition of Memantine HCl and Hydroxypropyl β-Cyclodextrin (Kleptose HPB). The mixture was allowed to stir overnight before casting the film on a Teflon surface using a BYK-Gardner film casting knife (Columbia, Md.). The film was dried in oven at 50° C. for 1 hour till completely dried. The films were then cut to size so that each piece contained a dose ranging from 2.5 mg to 30 mg. Tables 5 shows the resulting memantine orally dissolving film prepared using complexation process.

TABLE 5
Orally Dissolving Film Prepared with Complexed Memantine

| Ingredient | Function | 3 mg | 6 mg |
|---|---|---|---|
| Memantine HCl | Active | 3.0 | 6 |
| Hydroxypropyl | Taste-Masking/ | 37.5 | 75 |
| β-Cyclodextrin | Complexing agent | | |
| Polyvinyl | Film Forming Agent | 131.25 | 262.5 |
| Pyrrolidone | | | |
| Ethanol (Evaporated | Solvent | QS | QS |
| during processing) | | | |
| Flavors, | | QS | QS |
| Sweeteners* | | | |
| Total | | 171.75 | 343.5 |

The orally dissolving film prepared with complexed memantine were tasted (n=2), without the individuals receiving a dose of the drug, to determine the effectiveness of taste masking. The bitterness of the drug was significantly reduced using the described approach.

The disintegration of the orally dissolving film was tested as described in the United States Pharmacopeia (USP, Rockville, Md.). The results showed that the tablets compressed between 3-5 kP disintegrated in about 15 seconds.

To form orally dissolving tablets with complexed memantine, the memantine was dissolved in about 50 g Ethanol with Hydroxypropyl β-Cyclodextrin (Kleptose HPB). The solvent was evaporated in a rotary evaporator, the resulting complex was removed from the flask and dried in an oven at 50° C. for half hour. Once dried the composition was ground to obtain fine particle size. The Hydroxypropyl β-Cyclodextrin/memantine complex, was then mixed with Mannitol and Aerosil for 5 minutes. Magnesium Stearate was then added and mixed for an additional 1 minute. The final composition was then compresses into 400 mg tablets with a hardness of 3-5 kp. See Table 6.

TABLE 6
Orally Dissolving Tablet Prepared with Complexed Memantine

| Ingredient | Function | 20 mg |
|---|---|---|
| Hydroxypropyl β- | Taste- | 277.6 |
| Cyclodextrin with | Masking/Complexing | |
| Memantine HCl | agent | |
| Mannitol | Filler | 36.4 |
| Sodium Starch | Disintegrant | 80.0 |
| Glycolate | | |
| Aerosil | Glidant | 2.0 |
| Magnesium Stearate | Lubricant | 4.0 |
| Total | | 400 |

The orally dissolving tablets prepared with complexed memantine were tasted (n=2), without the individuals receiving a dose of the drug, to determine the effectiveness of taste masking. The bitterness of the drug was significantly reduced using the described approach.

### Example 4

One of the major problems encountered in the use of coated materials is the fracture of the coat during further processing under stress conditions. For example, the compression force required to prepare a tablet may cause the polymer, e.g., Eudragit, HPMC to fracture directly exposing the drug product to the individual. Consequently, if the integrity of the coating is compromised during compression it will result in leaching out of the Memantine into the oral cavity.

Orally Disintegrating Tablets of Memantine HCl were produced that provided a taste-masked drug product with a desirable dissolution profile. Coated, taste-masked Memantine HCl granules as described in Example 2 were mixed with excipients such as filler (Mannitol), disintegrant (Sodium Starch Glycolate) and glidant (Colloidal Silicon Dioxide) in a V-blender (Patterson Kelly, East Stroudsburg, Pa.) for 20 minutes. A lubricant (Magnesium Stearate) was the added and the composition was mixed for another 2 minutes. The 20 mg composition was the compressed using a Korsch PH106 rotary tablet press at hardness of 3 to 5 kP. Tables 7 and 8 show the compositions of the orally dissolving tablets of memantine using this process.

TABLE 7
Memantine Orally Dissolving Tablets

| Ingredients | 5 mg | 10 mg | 15 mg | 20 mg | 30 mg |
|---|---|---|---|---|---|
| Mannitol | 50.1 | 100.2 | 150.3 | 200.4 | 300.6 |
| Coated Taste-Masked | 28.4 | 56.8 | 85.2 | 113.6 | 170.4 |
| Memantine HCl Granules | | | | | |
| (Memantine HCl) | | | | | |
| Sodium Starch Glycolate | 20.0 | 40.0 | 60.0 | 80.0 | 120.0 |
| Magnesium Stearate | 1.0 | 2.0 | 3.0 | 4.0 | 6.0 |
| Colloidal Silicon Dioxide | 0.5 | 1.0 | 1.5 | 2.0 | 3.0 |
| Total | 100.0 | 200.0 | 300.0 | 400.0 | 600.0 |

TABLE 8
Memantine Orally Dissolving Tablets

| Ingredients | 3 mg | 6 mg | 9 mg | 12 mg | 24 mg |
|---|---|---|---|---|---|
| Mannitol | 30.06 | 60.12 | 90.18 | 120.24 | 240.48 |
| Coated Taste-Masked | 17.04 | 34.08 | 51.12 | 68.16 | 136.32 |
| Memantine HCl Granules | | | | | |
| (Memantine HCl) | | | | | |
| Sodium Starch Glycolate | 12 | 24 | 36 | 48 | 96 |
| Magnesium Stearate | 0.6 | 1.2 | 1.8 | 2.4 | 4.8 |
| Colloidal Silicon Dioxide | 0.3 | 0.6 | 0.9 | 1.2 | 2.4 |
| Total | 60 | 120 | 180 | 240 | 480 |

The orally dissolving tablets (20 mg) were tasted (n=2), without the individuals receiving a dose of the drug, to determine the effectiveness of taste masking. The bitterness of the drug was not noticeable upon tasting showing that orally dissolving tablets of memantine were effectively taste-masked using the described approach.

The disintegration of the orally dissolving tablets was tested as described in the United States Pharmacopeia (USP, Rockville, Md.). The results showed that the tablets compressed between 3-5 kP disintegrated in about 24 seconds. The dissolution of an orally dissolving tablet containing 20 mg of memantine was tested in 900 ml pH 1.2 NaCl/HCl buffer solution using a 100 rpm basket. Table 9 shows that more than 80% of the tablets were dissolved in 15 minutes.

TABLE 9
Orally Dissolving Tablet Dissolution

| | Strength |
|---|---|
| | 20 mg |
| | Tablets Hardness (Kp) |
| Time (min.) | 3 to 5 kp |
| 15 | 91 |
| 30 | 92 |
| 45 | 89 |
| 60 | 100 |

### Example 5

An orally dissolving film comprising memantine has been prepared by dissolving polyethylene oxide in water followed by the addition of plasticizer (Polyethylene glycol), a sweetening agent (Acesulfam K, Thaumatin), a bitter-taste receptor blocking agent (MAG Mimic Wixon-Fontarome. St. Francis, Wis.), Sodium Citrate, Polyoxyl Castor Oil and flavoring agent (Lemon powder). The taste-masked Memantine HCl granules, as prepared in Example 2, were then added and mixed for about 30 minutes before casting the film on a Teflon surface using a BYK-Gardner film casting knife (Columbia, Md.). The film was dried in oven first at 80° C. for 15 minutes and then at 50° C. until dried. The films were then cut to size so that each piece contained a dose ranging from 2.5 mg to 80 mg. Tables 10 and 11 show the compositions of the orally dissolving films of memantine.

TABLE 10
Memantine Orally Dissolving Films

| Ingredient | Function | 3 mg | | 6 mg | | 9 mg | | 12 mg | | 24 mg | |
|---|---|---|---|---|---|---|---|---|---|---|---|
| Memantine Taste | Active | 17.2 | | 34.4 | | 51.6 | | 68.8 | | 137.6 | |
| Masked Granules | | | | | | | | | | | |
| Polyethylene Oxide | Film former | 32.2 | | 64.4 | | 96.6 | | 128.8 | | 257.6 | |
| (Mol wt = 200,000 and | | | | | | | | | | | |
| 100,000) | | | | | | | | | | | |
| Polyoxyl castor oil | Surfactant | 2.3 | | 4.6 | | 6.9 | | 9.2 | | 18.4 | |
| Polyethylene Glycol | Plasticizer | 2.1 | | 4.2 | | 6.3 | | 8.4 | | 16.8 | |
| 400 | | | | | | | | | | | |
| Acesulfam K | Sweetener | 8.2 | | 16.4 | | 24.6 | | 32.8 | | 65.6 | |
| Thaumatin | Sweetener | 0.5 | | 1.0 | | 1.5 | | 2.0 | | 4.0 | |
| MAG mimic | Sweetener | 1.0 | | 2.0 | | 3.0 | | 4.0 | | 8.0 | |
| Lemon powder | Flavoring | 14.5 | | 29.0 | | 43.5 | | 58 | | 116 | |
| | Agent | | | | | | | | | | |
| Sodium Citrate | Bitter taste | 1.0 | | 2.0 | | 3.0 | | 4.0 | | 8.0 | |
| | Receptor | | | | | | | | | | |
| | blocker | | | | | | | | | | |
| Water (evaporated | Solvent | QS | | QS | | QS | | QS | | QS | |
| during processing) | | | | | | | | | | | |
| Total | | 79.0 | mg | 158 | mg | 237 | mg | 316 | mg | 632 | mg |

TABLE 11
Memantine Orally Dissolving Films

| Ingredient | Function | 4 mg | 5 mg | | 10 mg | | 20 mg | |
|---|---|---|---|---|---|---|---|---|
| Memantine Taste Masked | Active | | 28.6 | | 57.2 | | 114.4 | |
| Granules containing | | | | | | | | |
| (Memantine HCl per strength) | | | | | | | | |
| Polyethylene Oxide | Film former | | 53.6 | | 107.2 | | 214.4 | |
| (Mol wt = 200,000 to 100,000) | | | | | | | | |
| Polyoxyl castor oil | Surfactant | | 3.9 | | 7.8 | | 15.6 | |
| Polyethylene Glycol 400 | Plasticizer | | 3.5 | | 7.0 | | 14.0 | |
| Acesulfam K | Sweetener | | 13.6 | | 27.2 | | 54.4 | |
| Thaumatin | Sweetener | | 0.9 | | 1.8 | | 3.6 | |
| MAG mimic | Flavoring | | 1.6 | | 3.2 | | 6.4 | |
| | Agent | | | | | | | |
| Lemon powder | Flavoring | | 24.2 | | 48.4 | | 96.8 | |
| | Agent | | | | | | | |
| Sodium Citrate | Bitter taste | | 1.6 | | 3.2 | | 6.4 | |
| | Receptor | | | | | | | |
| | blocker | | | | | | | |
| Water (Evaporated during | Solvent | | QS | | QS | | QS | |
| processing) | | | | | | | | |
| Total | | | 131.5 | mg | 263 | mg | 526 | mg |

The orally dissolving films (4 mg) were tasted (n=2), without the individuals receiving a dose of the drug, to determine the effectiveness of taste masking. The bitterness of the drug was not noticeable upon tasting showing that orally dissolving films of memantine were effectively taste-masked using the described approach.

The disintegration of the orally dissolving films was tested as described in the United States Pharmacopeia (USP, Rockville, Md.). The results showed that the films disintegrated in less than 30 seconds.

The dissolution of 4 mg orally dissolving films containing memantine was tested in 900 ml pH 1.2 NaCl/HCl buffer solution using a 100 rpm basket. Table 12 shows that more than 80% of the films were dissolved in 15 minutes.

TABLE 12
Orally Dissolving Film dissolution

| | Strength |
|---|---|
| Time (min.) | 4 mg |
| 15 | 102 |
| 30 | 98 |
| 45 | 103 |
| 60 | 100 |

### Example 6

An orally dissolving film comprising memantine and polyvinyl pyrrolidone has been prepared. Table 13 shows the composition of the prepared film and exemplary ranges that may be used to produce other films. During the film casting process, the polyvinyl pyrrolidone (PVP K-90) polymer was dissolved in a portion of ethanol. Memantine, Lutrol E400 and Cremophor RH 40 were dissolved in a separate solution of ethanol. The solutions were then mixed and allowed to stand to allow deaeration. A film was then cast on a Teflon surface using a BYK-Gardner Film Casting knife. The cast film was then dried at 50° C. for about 90 minutes.

A second film was prepared without memantine using the same procedure to evaluate its dissolution properties. The prepared film was administered to four subjects and all four observed that the film dissolved in the mouth in less than 30 seconds. Accordingly, the dissolution of the memantine orally dissolving films should meet the criteria of memantine immediate release tablets of similar strength. Moreover, the memantine orally dissolving films of the present invention should provide the same bioavailability as that of memantine immediate release tablets and memantine solutions of similar strength.

TABLE 13

| | | | Film |
|---|---|---|---|
| | | % w/w Solids | composition |
| Ingredient | % w/w Solids | Preferred Range | (mg/100 mg) |
| Memantine | 1-50 | 5-30 | 24 |
| PVP K-90 | 20-90 | 40-80 | 71.2 |
| PEG 400 | 0.0-30.0 | 0.0-10.0 | 2.4 |
| (Lutrol E400) | | | |
| Cremophor | 0.1-10.0 | 1.0-5.0 | 2.4 |
| RH40 | | | |
| Ethanol* | qs | qs | qs |
| Total | 100 | 100 | 100 |

*The solvent is removed during the drying step

### Example 7

An orally dissolving film comprising memantine and polyethylene oxide has been prepared. Table 14 shows the composition of the prepared film and exemplary ranges that may be used to produce other films. During the film casting process, polyvinyl pyrrolidone and polyethylene oxide were dissolved in ethanol. Memantine, Lutrol E400 and Tween 80 were dissolved in a separate solution of ethanol. The solutions were then mixed and allowed to stand to allow deaeration. A film was then cast on a Teflon surface using a BYK-Gardner Film Casting knife. The cast film was then dried at 50° C. for about 90 minutes.

A second film was prepared without memantine using the same procedure to evaluate its dissolution properties. The prepared film was administered orally and dissolved in the mouth in less than 30 seconds. Accordingly, the dissolution of the memantine orally dissolving films should meet the criteria of memantine immediate release tablets of similar strength. Moreover, the memantine orally dissolving films of the present invention should provide the same bioavailability as that of memantine immediate release tablets and memantine solutions of similar strength.

TABLE 14

| | | % w/w Solids | Film |
|---|---|---|---|
| | | Preferred | composition |
| Ingredients | % w/w Solids | Range | (mg/100 mg) |
| Memantine HCl | 1-50 | 5-30 | 15.4 |
| Polyethylene | 1-90 | 10-70 | 61.5 |
| Oxide | | | |
| PVP | 0-90 | 0-50 | 15.4 |
| PEG 400 | 0-30.0 | 0-10.0 | 2.6 |
| (Lutrol E400) | | | |
| Tween 80 | 0-10.0 | 1.0-5.0 | 5.1 |
| Water* | qs | qs | qs |
| Total | 100 | 100 | 100 |

*The solvent is removed during the drying step

### Example 8

After oral administration of immediate release tablets memantine is completely absorbed (absolute bioavailability of approximately 100%). See Table 15. Memantine HCl is highly soluble and has been classified as a highly soluble and highly permeable drug. Therefore if properly formulated to have a substantially 100% dissolution, an orally dissolving formulation, e.g., tablets (ODTs) and films (ODFs), may qualify for a waiver of any studies to show bioavailability and bioequivalence. See “Waiver of In Vivo Bioavailability and Bioequivalence Studies for Immediate-Release Solid Oral Dosage Forms Based on a Biopharmaceutics Classification System”, U.S. Department of Health and Human Services, Food and Drug Administration.

TABLE 15
Dose Proportional Formulations

| Memantine HCl | | | | | | | | |
|---|---|---|---|---|---|---|---|---|
| (Needle Shaped | | | | | | | | |
| Crystals) | 2.5 mg | 5 mg | 10 mg | 15 mg | 20 mg | 40 mg | 60 mg | 80 mg |
| Microcrystalline | 48.8 | 97.5 | 195.0 | 292.5 | 390.0 | 780.0 | 1170.0 | 1560.0 |
| Cellulose (Prosolv)* | | | | | | | | |
| Croscarmellose Sodium | 1.1 | 2.2 | 4.4 | 6.6 | 8.8 | 17.6 | 26.4 | 35.2 |
| Talc | 2.5 | 5.0 | 10.0 | 15.0 | 20.0 | 40.0 | 60.0 | 80.0 |
| Mg stearate | 0.2 | 0.3 | 0.6 | 0.9 | 1.2 | 2.4 | 3.6 | 4.8 |
| Total Core Tablet* | 55.0 | 110.0 | 220.0 | 330.0 | 440.0 | 880.0 | 1320.0 | 1760.0 |
| Coating Opadry | 1.7 | 3.3 | 6.6 | 9.9 | 13.2 | 26.4 | 39.6 | 52.8 |
| (Containing HPMC) | | | | | | | | |
| Total coated | 56.7 | 113.3 | 226.6 | 339.9 | 453.2 | 906.4 | 1359.6 | 1812.8 |

*Core weight may be adjusted with fillers to +/−10% depending on filler densities; Prosolv is a mixture of microcrystalline cellulose and colloidal silicone dioxide

The dissolution of immediate release memantine HCl coated tablets was tested in 900 ml pH 1.2 NaCl/HCl buffer solution using a 100 rpm basket. Table 16 shows that more than 80% of the tablets was dissolved in 15 minutes.

TABLE 16
Immediate Release Tablet dissolution

Strength

| | | | |
|---|---|---|---|
| 5 mg | 10 mg | 15 mg | 20 mg |

Core tablets
Hardness (Kp)

| | | | |
|---|---|---|---|
| 4-10 | 7-13 | 10-16 | 12-20 |

| | |
|---|---|
| Time (min.) | % Dissolved |

| | | | | |
|---|---|---|---|---|
| 15 | 96 | 92 | 94 | 96 |
| 30 | 98 | 99 | 97 | 101 |
| 45 | 97 | 98 | 97 | 102 |

The dissolution of orally dissolving tablets and orally dissolving films were tested in biorelevant dissolution media simulating fasted and fed states (M. Marques, United States Pharmacopeia, Rockville, Md., in Dissolution Technology, May 2004) The dissolution of the orally dissolving formulations is the same as the immediate release tablets, i.e., more than 80% dissolved in 15 minutes.

In particular, the dissolution of memantine HCl orally dissolving formulations in three different BioRelevant Media were tested: (1) 900 ml pH 1.2 NaCl/HCl buffer, basket 100 rpm; (2) Fed State Simulated Intestinal Fluid (FESSIF) pH 5.0; and (3) Fasted State Simulated Intestinal Fluid (FASSIF): pH 6.5. The dissolution data for the orally dissolving tablets is shown in Table 17. The tablets prepared as described in Example 3, except the disintegrant level of sodium starch glycolate, was lower in this batch at 10% w/w in place of 20%, and dissolution values have been corrected based on assay values. The dissolution data for the orally dissolving films is shown in Table 18. The slight lag in dissolution at 15 minutes interval at pH above 5 is expected based on the properties of Eudragit E polymer.

TABLE 17
Dissolution of Orally Dissolving Tablets

| | Strength | | |
|---|---|---|---|
| | 20 mg | 20 mg | 20 mg |
| | Media | | |
| | (1) | (2) | (3) |
| Time (min.) | % Dissolved | | |
| 15 | 91 | 82 | 87 |
| 30 | 92 | 94 | 93 |
| 45 | 89 | 96 | 97 |
| 60 | 100 | 100 | 100 |

TABLE 18
Dissolution of Orally Dissolving Films

| | Strength | | |
|---|---|---|---|
| | 10.8 | 10.8 mg | 10.8 mg |
| | Media | | |
| | (1) | (2) | (3) |
| Time (min.) | % Dissolved | | |
| 15 | 102 | 80 | 77 |
| 30 | 98 | 93 | 93 |
| 45 | 103 | 97 | 97 |
| 60 | 100 | 100 | 100 |

Thus, the dissolution of the orally dissolving formulations of the present invention in pH 1.2 NaCl/HCl buffer, i.e., stomach pH, is more than 80% in 15 minutes and may be more than 85% in 15 minutes. Therefore, the bioavailability, and pharmokinetic parameters, of the orally dissolving formulations, e.g., tablets (ODTs) and films (ODFs), are approximately the same as the immediate release memantine HCl coated tablets. The pharmokinetic parameters determined for patients receiving two 20 mg immediate release tablets (i.e., a single 40 mg dose of memantine) is shown in Table 19. The pharmokinetic parameters are disclosed in U.S. Patent Publication No. 2007/0065512, the disclosure of which is hereby incorporated by reference in its entirety.

TABLE 19
Pk Parameters for IR Tablets of Memantine

| Parameter | Treatment A |
|---|---|
| Cₘₐₓ (ng/mL) | 59.83 ± 12.91 |
| Tₘₐₓ (h) | 6.1 ± 1.3 |
| AUC₀₋ₜ (ng · h/mL) | 4522 ± 801 |
| AUC_{0-∞} (ng · h/mL) | 4653 ± 830 |
| T_{1/2} (h) | 64.10 ± 10.39 |

Accordingly, the pharmokinetic parameters for the orally dissolving formulations of the present invention may be estimated as follows: the time to maximum plasma concentrations (Tₘₐₓ) following oral doses of an ODF an ODT with 2.5 to 40 mg of memantine ranges between 3 and 7 hours, with an elimination half-life (T_{1/2}) of approximately 60-80 hours. The peak plasma concentrations (Cₘₐₓ) after administration of a single 20 mg ODT or ODF would range from about 22 to about 46 ng/mL. The area under the plasma concentration-time curve (AUC₀₋ₜ and AUC_{0-∞}) after administration of a single 20 mg ODT or ODF would range from about 2000 to about 2500 ng·h/mL. The AUC and Cₘₐₓ values of memantine, however, increase proportionally with dosages over the range of 5 to 40 mg. Thus, one skilled in the art with the benefit of this disclosure may readily determine pharmokinetic parameters for any specific dosage of memantine used in a particular orally dissolving formulation.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

1. An orally dissolving formulation comprising at least one water soluble polymer and memantine or a pharmaceutically acceptable salt thereof.

2. The orally dissolving formulation of claim 1, wherein the water soluble polymer is selected from the group consisting of methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, agar, carrageenan, xanthan gum, tragacanth, guar gum, acacia gum, arabic gum, polyethylene glycol, polyethylene oxide, polyvinyl pyrrolidone, polyvinyl alcohol, cyclodextrins, carboxyvinyl polymers, sodium alginate, polyacrylic acid, methylmethacrylate and mixtures thereof.

3. The orally dissolving formulation of claim 1, further comprising a taste masking agent, a flavoring agent, a softener, a diluent, a stabilizer, a dye, a colorant, a disintegrant, an excipient, or combinations thereof.

4. The orally dissolving formulation of claim 1, wherein the formulation is a film or a tablet.

5. (canceled)

6. The orally dissolving formulation of claim 1, wherein the memantine is taste-masked.

7. The orally dissolving formulation of claim 1, wherein the dissolution rate of the active ingredient is more than about 80% within about the first 15 minutes following entry of the dosage form into a use environment.

8. The orally dissolving formulation of claim 1, wherein the dissolution rate of the active ingredient is more than about 85% within about the first 15 minutes following entry of the dosage form into a use environment.

9. The orally dissolving formulation of claim 1, wherein the disintegration rate of formulation is less than 30 seconds following entry of the dosage form into a use environment.

10. The orally dissolving formulation of claim 1, wherein the disintegration rate of formulation is less than 15 seconds following entry of the dosage form into a use environment.

11. The orally dissolving formulation of claim 1, wherein the formulation comprises 2.5 to 40 mg of memantine or a salt thereof and provides an in vivo plasma profile comprising:
a mean Tmax of about 4 or more hours;
a mean Cmax of less than about 100 ng/ml; and
a mean AUC_{0-∞}) of more than about 250 ng h/ml.

12. An orally dissolving formulation comprising memantine or a salt thereof, wherein the formulation comprises 2.5 to 40 mg of memantine or a salt thereof and provides an in vivo plasma profile comprising:
a mean Tmax of about 4 or more hours;
a mean Cmax of less than about 100 ng/ml; and
a mean AUC_{0-∞} of more than about 250 ng h/ml.

13. The orally dissolving formulation of claim 12, wherein the formulation is a film or a tablet.

14. (canceled)

15. (canceled)

16. The orally dissolving formulation of claim 12, wherein the dissolution rate of the active ingredient is more than about 80% within about the first 15 minutes following entry of the dosage form into a use environment.

17. The orally dissolving formulation of claim 12, wherein the dissolution rate of the active ingredient is more than about 85% within about the first 15 minutes following entry of the dosage form into a use environment.

18. The orally dissolving formulation of claim 12, wherein the disintegration rate of formulation is less than 30 seconds following entry of the dosage form into a use environment.

19. The orally dissolving formulation of claim 12, wherein the mean Cmax is less than about 60 ng/ml.

20. The orally dissolving formulation of claim 12, wherein the mean Cmax is less than about 10 ng/ml.

21. The orally dissolving formulation of claim 12, wherein the mean AUC_{0-∞} is less than about 750 ng h/ml.

22. The orally dissolving formulation of claim 12, wherein the mean AUC_{0-∞} is less than about 400 ng h/ml.

23. The orally dissolving formulation of claim 12, wherein the formulation comprises 6 mg of memantine or a salt thereof and provides an in vivo plasma profile comprising:
a mean Tmax of about 4 or more hours;
a mean Cmax of between about 6 to 12 ng/ml; and
a mean AUC_{0-∞} of between about 500 to 1000 ng h/ml.

24. The orally dissolving formulation of claim 12, wherein the formulation comprises 3 mg of memantine or a salt thereof and provides an in vivo plasma profile comprising:
a mean Tmax of about 4 or more hours;
a mean Cmax of between about 3.5 to 5.5 ng/ml; and
a mean AUC_{0-∞} of between about 250 to 450 ng h/ml.

25. A method for treating a disorder of the central nervous system, comprising administering to a patient in need thereof an orally dissolving formulation comprising at least one water soluble polymer and memantine or a pharmaceutically acceptable salt thereof.

26. The method of claim 25, wherein the disorder of the central nervous system is Alzheimer's Disease.

27. A method for treating a childhood behavioral disorder, comprising administering to a patient in need thereof an orally dissolving formulation comprising at least one water soluble polymer and memantine or a pharmaceutically acceptable salt thereof.

28. The method of claim 27, wherein the childhood behavioral disorder is autism.
